(19) United States
(12) Patent Application Publication
(10) Pub. No.: US 2025/0261886 A1
(43) Pub. Date: Aug. 21, 2025

(54) Systems and Methods for Integrating Eye Gaze Tracking Into A Multimodal Dialog Agent for Remote Patient Assessment

(71) Applicant: Modality.AI, San Francisco, CA (US)

(72) Inventors: Daniel Tisdale, San Luis Obispo, CA (US); Jackson Liscombe, Mill River, MA (US); David Pautler, Sarasota, FL (US); Vikram Ramanarayanan, San Francisco, CA (US)

(21) Appl. No.: 18/582,442

(22) Filed: Feb. 20, 2024

(51) Int. Cl. A61B 5/16; A61B 3/14; G06T 7/00; G06T 7/20; G06T 7/70; G16H 40/67

(52) U.S. Cl. CPC A61B 5/163; A61B 3/14; G06T 7/0012; G06T 7/20; G06T 7/70; G16H 40/67; G06T 2200/24; G06T 2207/30041; G06T 2207/30201

(57) ABSTRACT

A system for the remote patient assessment and monitoring system that collects and analyzes eye-gaze information for the purposes of identifying users that show symptoms of cognitive impairment. The system administers tasks to a user via the user's computing device. A camera captures the performance of the tasks by the user. The system then verifies synchronization and then analyzes the captured performance image data to determine a patient status for the user. The system applies calibration and normalization processes to ensure that the tasks are properly presented and that the user data can be used regardless of changes to resolution, hardware, display or other factors.

### FIELD OF THE INVENTION

The field of the invention is remote diagnostics and patient care.

### BACKGROUND

The background description includes information that may be useful in understanding the present invention. It is not an admission that any of the information provided herein is prior art or relevant to the presently claimed invention, or that any publication specifically or implicitly referenced is prior art.

Abnormalities in eye gaze metrics have been clinically validated for many diseases, such as multiple sclerosis, AIDS dementia complex, antisocial personality disorder, autism spectrum disorder, schizophrenia, psychosis, dyslexia, eating disorders, social anxiety disorders, attention deficit hyperactivity disorder, fetal alcohol spectrum disorder, Parkinson's disease, and bipolar disorder.

Therefore, there is a well-established relationship between eye gaze data and cognitive and neurological functioning. Several tasks (and associated metrics) derived from in-clinic eye gaze assessment protocols can capture this relationship and its breakdown. For instance, a saccade is the rapid movement the eyes do simultaneously to change the line of sight. Smooth pursuit eye movements are the voluntary tracking performed when stabilizing gaze on a moving visual target. Fixations are the stationary states of the eyes during which eye gaze is held upon a specific location in the visual scene. Fixations can be furthermore incorporated into saliency metrics based on models of human attention to certain locations in a video or picture. Finally, the entire path of a gaze sequence, or scan path, for a particular task can be considered as either a shape in and of itself or as input into machine learning algorithms. Such metrics derived from eye gaze movements have been shown to correlate with several cognitive and neurological disorders, both degenerative and developmental.

While in-person clinical administration of eye-gaze tests has helped detect the potential presence of certain diseases in patients, the administration of tasks in a telemedicine setting creates new challenges and obstacles that are not present in the in-person setting. An in-person clinician does not have to account for obstacles such as network discrepancies, lag, desynchronization, variances in hardware, or other variables. When a clinician is in a room with a patient administering the tests, the clinician can simply observe and make their findings.

Moreover, patients for whom eye-gaze tests are particular necessary (e.g., those with Parkinsons-related dementia) may not be ambulatory or otherwise be able to physically go to a clinic or doctor's office. In these cases, being able to accurately attend and assess their conditions remotely is crucial.

Thus, there is still a need for a remote system for patient assessment that integrates eye gaze tracking while overcoming the obstacles unique to such a system.

### SUMMARY OF THE INVENTION

The inventive subject matter provides apparatus, systems and methods in which a computer system engages patients in an interactive dialog session, via a virtual agent, and guides patients through several spoken, orofacial, cognitive, and gaze tasks inspired by clinical protocols. The dialog protocol includes a selection of exercises that have been widely used in oculomotor pathology research as well as in-person clinical practice, including: smooth pursuit, saccade, free image exploration, directed image exploration, and the congruent and incongruent Stroop tests. As discussed herein, the system computes eye gaze metrics relevant to the assessment of their overall neurological and mental health. These can be combined with Applicant's work in deriving speech, facial, linguistic, and motoric metrics relevant to the assessment of their overall neurological and mental health. This makes it possible to provide a comprehensive assessment of a person's neurological and mental health remotely.

The system of the inventive subject matter includes a server computer system that includes a database populated with a plurality of visual tasks. The server computer system executes visual tasks and a virtual agent that are presented to a user on the user's computing device. The visual tasks are tasks that are designed to elicit eye-gaze and eye-movement responses from the user that are then captured in image data.

At the user premises, a camera (e.g., a camera communicatively coupled with the user's computing device) captures the image data containing the performance of the visual tasks by the user (which includes the eye-gaze and other eye-related movements elicited by the visual tasks). The image data is transmitted back to the server, which derives metrics from the execution of the visual tasks. The metrics can then be used by the server to determine whether a user is exhibiting symptoms of a condition such as mild neurocognitive disorder (“MND”) and/or mild cognitive impairment (“MCI”).

As a part of the process, the server provides a calibration task that is presented via the user computer device. The calibration task enables the system to calibrate the session to account for variables such as a user's computer's display size, camera field of view, user distance from the camera, and to properly correlate the user's gaze as observed from the camera image data to on-screen positions.

The server and/or the user's computing device can also execute normalization processes that can help normalize the gathered data such that it can be used and recalled independently of changes in resolution, window size, or even hardware from one task to the next or from one session to the next.

It is contemplated that the systems and methods of the inventive subject matter can be used in combination with Applicant's work in using other modalities (e.g., voice, dialog, movement, etc.) in remote patient assessment systems. The following applications are incorporated herein by reference in their entirety: U.S. patent application Ser. No. 17/471,929 filed Sep. 10, 2020 titled “Use of Virtual Agent to Assess Psychological and Medical Conditions; U.S. patent application Ser. No. 17/508,693 filed Oct. 22, 2021 titled “Multimodal Conversational Platform for Remote Patient Diagnosis and Monitoring”; U.S. patent application Ser. No. 17/552,351 filed Dec. 15, 2021 titled “Remote Monitoring of Respiratory Function Using a Cloud-Based Multimodal Dialogue System”; U.S. patent application Ser. No. 17/724,320 filed Apr. 19, 2022 titled “Customizing Computer Generated Dialog for Different Pathologies”; U.S. patent application Ser. No. 17/974,306 filed Oct. 26, 2022 titled “Multimodal Dialog-Based Remote Patient Monitoring of Motor Function”; U.S. patent application Ser. No. 18/130,135 filed Apr. 3, 2023 “Systems and Methods for Remotely Assessing A Condition in a Patient”; U.S. patent application Ser. No. 18/383,259 filed Oct. 24, 2024 titled “Multimodal Investigation of Speech, Text, Cognitive and Facial Video Features for Characterizing Depression With and Without Medication.”

Various objects, features, aspects and advantages of the inventive subject matter will become more apparent from the following detailed description of preferred embodiments, along with the accompanying drawing figures in which like numerals represent like components.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### BRIEF DESCRIPTION OF THE DRAWING

FIG. 1 is an overview of the system, according to embodiments of the inventive subject matter.

FIG. 2 provides a flowchart illustrating the processes executed according to embodiments of the inventive subject matter.

FIG. 3 provides an illustrative example of the screen presented during the calibration task, according to embodiments of the inventive subject matter.

FIG. 4A is a flowchart illustrating the normalization process of step 220 in greater detail, according to embodiments of the inventive subject matter.

FIG. 4B is an illustrative example of a screen shown at step 221, according to embodiments of the inventive subject matter.

FIG. 5A provides an illustrative example of a horizontal line subtask, according to embodiments of the inventive subject matter.

FIG. 5B provides an illustrative example of a circular path subtask, according to embodiments of the inventive subject matter.

FIG. 6 shows an illustrative example of the presentation of the saccade task on the screen of the user computing device, according to embodiments of the inventive subject matter.

FIG. 7 shows the presentation of the congruent Stroop task on the screen of the user computing device, according to embodiments of the inventive subject matter.

FIG. 8 shows the presentation of the incongruent Stroop task on the screen of the user computing device, according to embodiments of the inventive subject matter

FIG. 9 shows an illustrative example of the presentation of the directed image exploration task depicting an image, according to embodiments of the inventive subject matter.

FIG. 10 is a flowchart illustrating the processing of live processing data, according to embodiments of the inventive subject matter.

FIG. 11 is a flowchart illustrating the synchronization process of step 260, according to embodiments of the inventive subject matter.

FIG. 12 is a flowchart illustrating the processing of the generated metrics to derive a user status, according to embodiments of the inventive subject matter.

### DETAILED DESCRIPTION

Throughout the following discussion, numerous references will be made regarding servers, services, interfaces, engines, modules, clients, peers, portals, platforms, or other systems formed from computing devices. It should be appreciated that the use of such terms, is deemed to represent one or more computing devices having at least one processor (e.g., ASIC, FPGA, DSP, x86, ARM, ColdFire, GPU, multi-core processors, etc.) programmed to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable media storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, the Internet, LAN, WAN, VPN, or other type of packet switched network.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

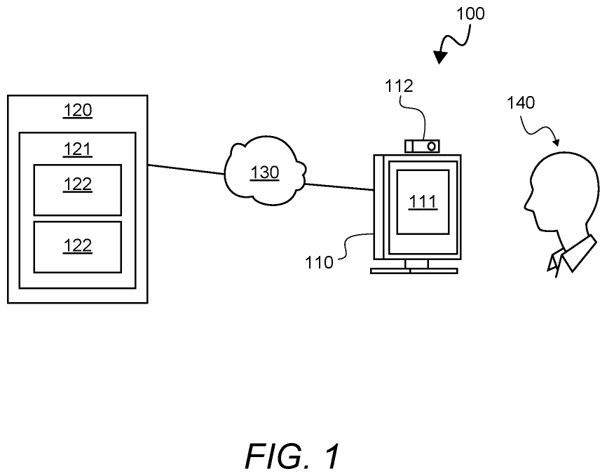

FIG. 1 is an overview of the system 100, according to embodiments of the inventive subject matter.

The system 100 includes a remote computing device 120 (otherwise referred to server 120) that can communicate to one or more client devices 110 over a network 130 (e.g., the internet). The server 120 can be one or more computing devices that include at least one processor, storage, and communication interface(s), located in one or more locations that can store and communicate data with other components of the system 100. The server 120 can include a database 121 that stores a plurality of performance tests or tasks 122.

The tasks 122 include computer executable instructions that enable the system 100 to administer a task to a user, obtain performance information captured via one or more sensors (e.g., a camera, microphone, etc.), and then enable the server 120 to analyze the performance of the test and determine whether a condition may exist.

For a given task 122, the database 121 stores the executable instructions that enables the presentation of instructions via a virtual agent 111 (which could be a video of someone performing the test), the capturing of the patient/user 140 performing the test (such as via a video camera 112 connected or integral to the computing device 110), the analysis of the task to determine a condition (in this case, level of impairment) and the transmission of the test to appropriate parties (the patient themselves, health care providers, etc.).

The data and instructions associated with a task 122 can include one or more metrics that are associated with the task 122 that can give an indication of the potential presence of mild neurocognitive disorder (“MND”) and/or mild cognitive impairment (“MCI”) or other cognitive impairments such as Alzheimer's or other related dementias, as well as symptoms thereof and the severity. The metrics can be thought of as the measurable characteristics associated with the user's performance of the task that have been observed to be related or correlated with MND/MCI. The metrics thus could be considered attributes, whose values can be measured by the system when the user performs a task. The data and instructions of task 122 can also include one or more thresholds of values, beyond which (above or below, depending on the metric) the metric can be considered to be indicative of the presence of autism spectrum disorder (alone or in combination with other metrics).

The client computing devices 110 can access the functions of the inventive subject matter via multiple ways. For example, a downloadable application or via a web portal accessible over a browser. The client computing devices 110 include at least one processor, at least one non-transitory computer-readable storage medium, and I/O interfaces that allow a user to receive data from and interact with the computing device 110 (e.g., monitor, touch screen, speakers, mouse, keyboard, cameras, etc.). The client computing devices 110 also have communication interfaces (e.g., Wi-Fi, wired internet connection, cellular, etc.) that enable the device 121 to exchange data over network 130. Examples of suitable computing devices 110 can include desktop computers, laptop computers, tablets, smartphones, and video game consoles.

To administer the tasks 122 and enable other interactions with a patient, a client computing device 120 executes a virtual agent 111. The virtual agent 111 can be installed on the client computing device 110. In other embodiments, the virtual agent 111 is executed by the server 120 and merely presented on the client computing device 110 via a web browser or other user-facing portal.

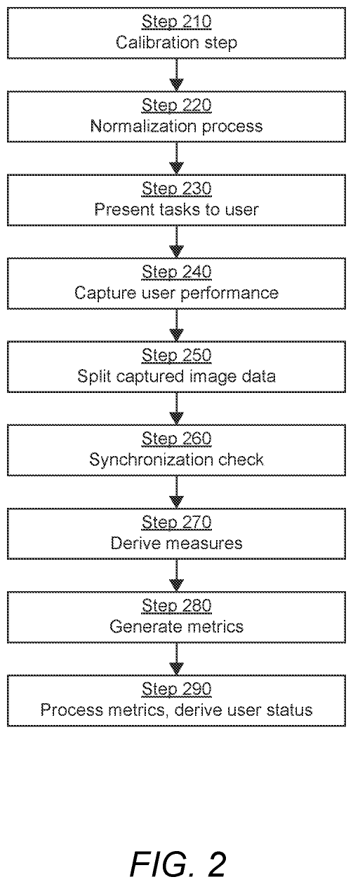

FIG. 2 provides a flowchart illustrating the processes executed according to embodiments of the inventive subject matter.

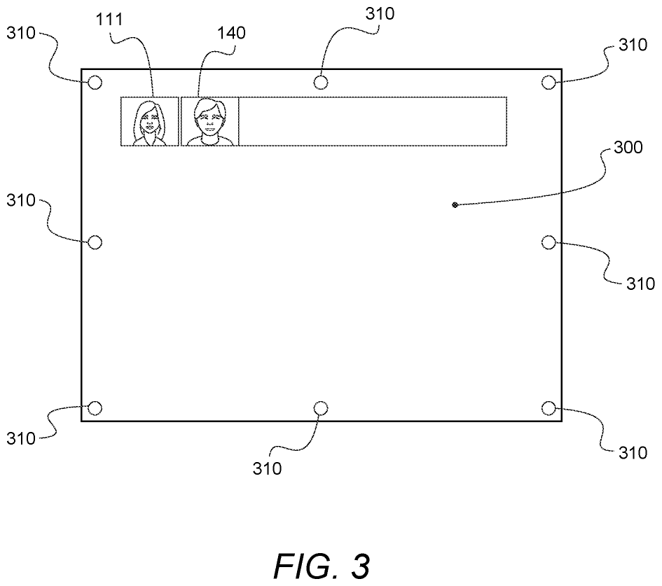

Step 210 of the process is a calibration task. FIG. 3 provides an exemplary screenshot of the screen presented during the calibration task.

At the calibration step, the virtual agent 111 presents, via the user's screen, points/buttons 310 or other visual indicators at or close to the edges of the display of the user's computing device 110. The buttons 130 can be presented sequentially, such that the user 140 follows and clicks on the buttons with their mouse cursor from one button to the next. Alternatively, the buttons 130 can be presented all at once with an indication of a sequence (such as via a button lighting up to indicate it is to be pressed next, an audio instruction, etc.). The buttons 310 can be round or can be any other shape or color.

As the user performs the calibration task, image data captured with the camera 112 of the computing device 110 depicts the changes in eye gaze/direction while the user finds each button on the screen and then moves the cursor to that button for clicking.

The user computing device 110 and/or the server 120 then correlates an elapsed time of each click with a corresponding frame of the captured image data. The image data of the calibration is processed by at least one of the user computing device 110 and/or the server 120 with software such as WebGazer to estimate an eye gaze direction at each point. In other embodiments, specialized hardware can be used to more precisely track eye gaze throughout the processes discussed here. However, preferred embodiments employ the software solutions discussed herein one of the advantages of the systems and methods of the inventive subject matter is that the system can be deployed and used with patients that do not have access to specialized hardware and, in many cases, only have basic computing devices to begin with.

Based on the correlation of the clicks and the user's estimated eye gaze, the server 120 and/or the user computing device 110 determines the boundaries of the usable area within the display of the user computing device 110 (typically, within a browser or installed application running on the user computing device 110). The usable area is the area within which the server 120 will present, via the browser/application window and with the virtual agent 111, the tasks discussed herein for performance by the user.

During calibration, the user is asked to click dots on the edge of the screen. As they point to click the dots with a mouse cursor, the eye gaze model executed by the server 120 uses the cursor as it clicks as the “ground truth” for gaze location to calibrate the model to facial landmarks.

The correlation of the clicks and the users estimated eye gaze provides a calibration for the system because the system will have data establishing the edges of the usable area and can also estimate in-between points for both eye gaze and coordinates within a usable area.

In the example of FIG. 3, the virtual agent 111 is also visually depicted with an avatar that can be animated to speak the instructions and provide an example execution of the task. The example of FIG. 3 also shows the view of the user 140 in a window. This can provide guidance to the user 140 on proper framing. In embodiments, the virtual agent 111 can, verbally and/or via text, provide guidance to the user 140 to move relative to the camera for optimal framing, as discussed in further detail below.

The cursor is only used during the calibration stage of step 210. After that, only the user's eyes are tracked via the capture of image data and subsequent processing as discussed further below.

At step 220, the system 100 performs a normalization process to account for variances in hardware capabilities across the system 100, including at the user's computer device 110. The normalization process is designed to maintain relatively normalized speeds and sizes in presentation and information acquisition across different types of computer hardware. The objects of interest presented in the tasks, the dynamically drawn browser vector objects, the images and videos presented and captured are adjusted relative to the window size, resolution and refresh rate with fixed minimums and maximums to ensure the widest possible field of view for the user's gaze while staying within an expected window of quality. Other information that the server 120 can obtain from the user computing device 110 includes screen size, refresh rate, overall monitor resolution, CPU information, graphics capabilities, etc. This additional information can be used by the server 120 to further produce a more seamless experience with the presentation of the tasks 122 and the collection of the image data from camera 112 and audio data (where applicable) from a microphone.

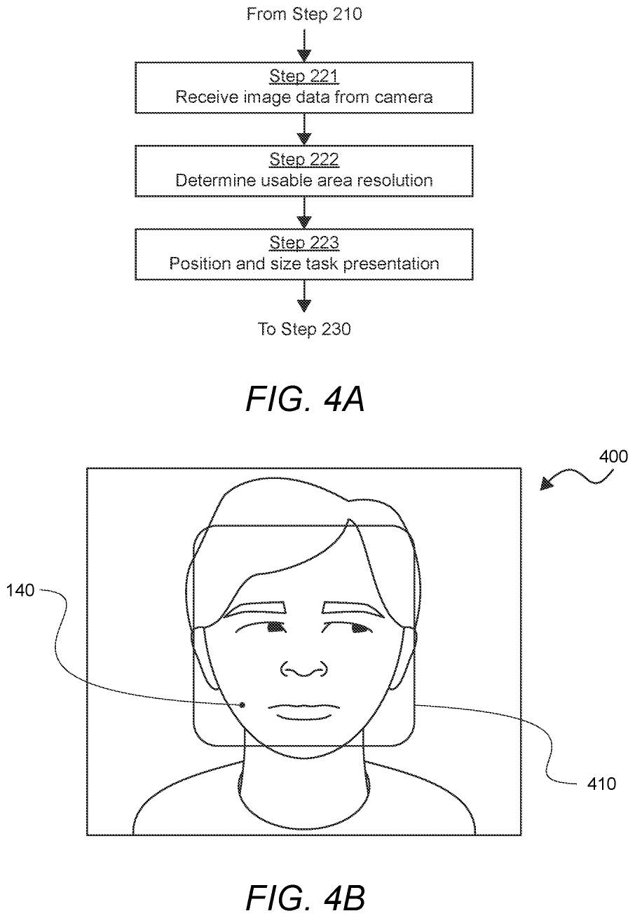

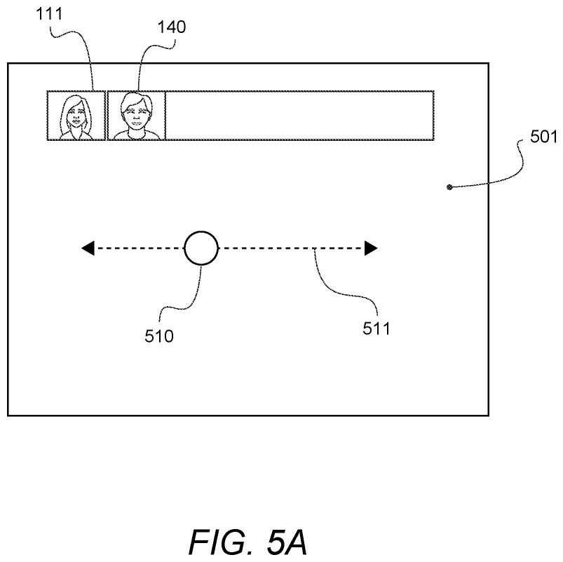

FIG. 4A is a flowchart illustrating the normalization process of step 220 in greater detail. The process below is described as originating from the server 120, but some or all of the steps can be performed by the user computing device 110, with the executable instructions provided by the server 120 for executing by the user computing device 110.

At step 221, the server 120 receives the stream (i.e., the camera feed) from the camera 112. FIG. 4B shows the display 401 presented to the user at this step. The server 120 crops and mirrors the received stream and overlays a colored box 410. This feed, including the box 410 is displayed to the user on the computer device 110. The virtual agent 111 presents instructions for the user to fill the box 410 with their face as best they can. This results in the user's face being centered while maintaining an appropriate distance from the screen (for most display sizes, this is approximately 1.5-2.5 feet from the screen).

The box 410 is maintained over an image of the user 140 during the performance of the tasks 122. This way, the user 140 can make corrections as needed to ensure their face remains within the box 410 at the appropriate size.

At step 222, the server 120 determines a resolution of usable area/space within the display of computing device 110. The boundaries of the usable area were estimated in the calibration step 210 above, and the normalization task steps discussed herein help the system correctly fit and space the presentation of the tasks discussed herein within the boundaries established for the usable area. For embodiments where the virtual agent 111 and tasks 122 are accessed via a web browser, the usable space comprises the resolution of the browser window. In embodiments where the virtual agent 111 and the tasks 122 are accessed via a dedicated downloaded application, the usable space comprises the resolution of the displayed application within the computer screen.

In certain embodiments, the usable area is not determined at the calibration step-only a link is established between the clicking and the eye gaze for the purposes of establishing tracking boundaries. In these embodiments, the usable area is determined at step 222 based solely on the resolution of the browser window/application window as displayed on the display of the user computing device 110.

Each stored task 122 includes information about the sizing and spacing ratios, and necessary resolutions for the adequate presentation and executing of each task. Having obtained the resolution of usable space, the server 120 determines (for each task 122) the positioning of objects presented for the task, the display of object movement (if any) for the task, the measurements of eye movements by the user for the task, and sizes of objects and also measured features as ratios relative to the resolution of the usable area, all based on the stored information and determined usable area resolution.

For example, a dot appearing half-way up the screen minus 200 pixels to account for the information tab on top. In another example, objects presented move up to 5% away from the edge of the screen or to a certain number of pixels away from the center that would typically imply the use of an extra wide monitor.

This allows for the presentation of the tasks and the measurement of eye gaze and movement to be relatively consistent and pleasant viewing angles (e.g., objects do not go off-screen or disappear) across different screen sizes, resolutions and browser windows/application windows (even if resized between tasks). This consistency can be further be maintained if the user changes hardware (the camera 112, monitor, or entire computing device 120 from a laptop to a tablet or a laptop to a desktop computer, for example) between tasks or between sessions.

At step 223, the server 120 uses the determined resolution of usable space to, for each task 122, the positioning and size of the objects that are shown for the task based on established minimum and maximum resolutions. In embodiments of the inventive subject matter, the system is intended for use primarily in laptops, desktops and large tablets such that the positioning and sizing of the presentation of the tasks is best suited for displays with resolutions between 1280×720 to 3840×2160, with 1920×1080 being the primary resolution target. The fixed minimum and maximum resolutions guide the minimum and maximum for objects on screen, which maintain the consistent sizing and spacing between objects and preventing objects from being displayed incorrectly. In embodiments of the inventive subject matter, the server 120 is programmed to provide a message to the user's computing device 110 for presentation to the user 140 if the resolution of their monitor is outside of the minimum and maximum ranges. In embodiments of the inventive subject matter, the server 120 can account for a monitor or display having a greater resolution than the maximum by limiting the display area of the tasks 122 and other aspects of the system to an area having the maximum resolution.

The normalization process of FIG. 4B can be performed at the start of a session only, or can be performed before the performance of each task 122. In the event that a task 122 is not properly captured (e.g., due to interruptions, lag, or other technical difficulties), the normalization process of FIG. 4B can be re-executed before the task 122.

The effect of the normalization process can be illustrated in the following example:

The system presents a visual task that requires a particular eye movement. Based on the normalization process discussed above, the server 120 has established that the computing device 110 for this particular user has a certain usable area and that the usable area has a particular resolution. The presentation of the objects in the visual task are presented according to a priori known ratios of size and spacing for that resolution. When the user's eye movements are measured, the measured movement is relative to the presentation of the objects and (where applicable) movement of objects. Since the server 120 knows the actual locations of the objects as well as their ratios, the server 120 can then correlate the measured eye movements to the known ratios to establish a ratio for the measured eye movements as well (e.g., for a given resolution, a movement of X pixels in a certain amount of time or for a certain task corresponds to a movement of a proportional ratio within the usable area). If the user changes hardware and/or usable area size between sessions or between tasks, the stored captured data can be carried over to the new resolution of the new hardware or usable area size because the ratios of a task (and therefore the ratios of the captured eye movements) remains constant.

At step 230, the virtual agent 111 presents, via the user's computer device 120, one or more tasks 122 for performance by user 140.

The tasks 122 generally are tasks designed to elicit a certain kind of eye activity in the user 140 that can be measured via captured image data. Some tasks 122 involve only eye activity that is detected and analyzed from visual video data. Other tasks 112 can include the use of audio data such as words or other sounds uttered by the user 140 and captured by a microphone. The tasks 122 can include:

An extreme saccade task: This task involves exaggeratedly and rapidly looking back and forth while keeping the head still. For this task, the virtual agent 111 instructs the user 140 to rapidly move their eyes back and forth to look as far to each side as possible. In embodiments, the virtual agent 111 can show how the task is performed via movement of the avatar's eyes. This task can be performed for a predetermined amount of time, or until a certain amount of movements have been captured by the system 100. The camera 112 captures the movement of the user's eyes as depicted in the captured image data. The virtual agent 111 can then indicate a start to the task via a countdown to zero or other audio instruction and/or textual instruction and an end to the task at the end of a predetermined amount of time. The extreme saccade task does not actually display any objects on the screen as part of the task.

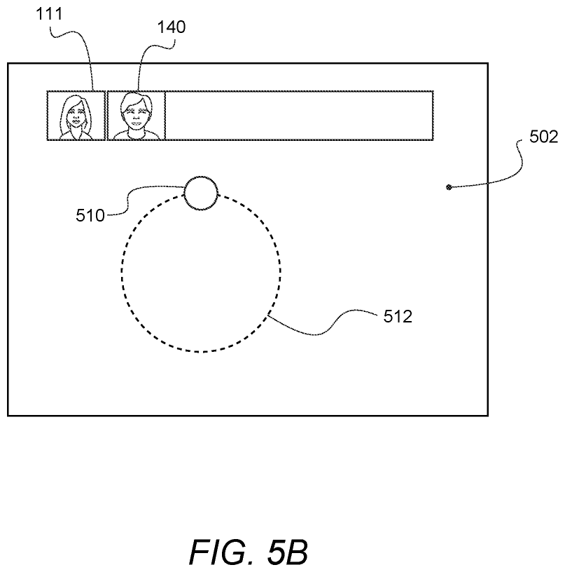

A smooth pursuit task: The smooth pursuit task comprises a task whereby the user 140 follows an object 510 on the screen moving a predetermined path (which can be a horizontal line and a circular path, or other pattern) while maintaining their eye-gaze on the object. FIGS. 5A and 5B illustrate this task. FIG. 5A illustrates a horizontal line subtask, whereby the object 510 displayed on screen 501 travels back and forth on a horizontal line 511. FIG. 5B illustrates a circular path subtask, whereby the object 510 displayed on screen 502 travels in a circular path 412. The lines 411, 412 are only on FIG. 4A-4B for illustration, and are not shown or otherwise visible to the user 140 during the execution of the test. The metrics derived from the smooth pursuit task (e.g., the horizontal line subtask and circular path subtask) can include metrics that reflect an accuracy of gaze-the ability of the user 140 to follow the object 510. This can be determined by the server 120 by determining the mean squared error or mean absolute deviation between the actual position of the object 510 at any given time during the exercise and the predicted eye gaze estimate at that same time during the exercise.

For the horizontal line subtask of FIG. 4A, the duration of the subtask can comprise a predetermined amount of movements of the object 410 along the path 411. For example, the length of the horizontal line subtask can comprise three cycles of the object 410 moving from one end of path 411 (e.g., the left side) to the other end (e.g., the right side) and then back to the first end (e.g., the left side again).

For the circular path subtask, the duration of the subtask can be a number of revolutions of the circle of path 412. For example, three revolutions.

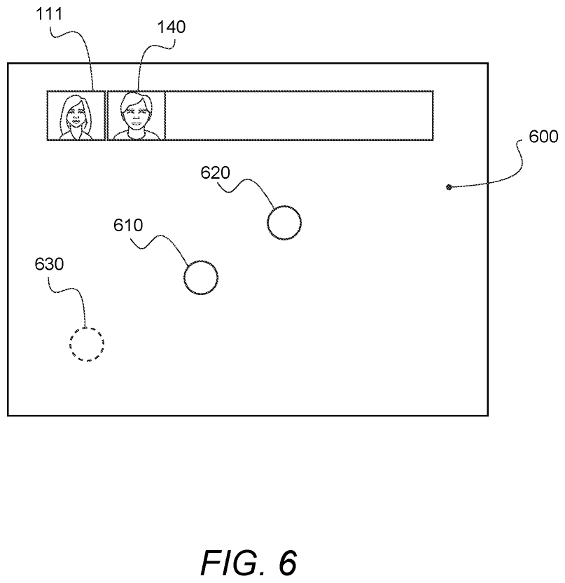

A saccade task: In the saccade task, the virtual agent 111 presents objects at random positions of the screen, and the user 140 must look in the directions of the objects as they appear. FIG. 6 shows an illustrative example of the presentation of the saccade task 500 on the screen of computing device 110. In the saccade task, the computing device 110 presents a first object 510, then a second object 520 at a different point in the screen, and then a third object 530 (yet to be shown, illustrated by dotted lines to show its location that is different from the other two objects 510, 520). The camera 112 captures the user's eye movements as each object is presented. The saccade task ends upon reaching the last object in a sequence and giving the user a pre-determined amount of time to direct their gaze toward the last object. Metrics measured for the saccade task can include an accuracy of gaze measured by the server 120 in the same way as the smooth pursuit task. Other metrics can include a measured speed of saccade whereby the server 120 determines the time it takes the user 140 to go from one object 510 to the next object 520 and so on during the task.

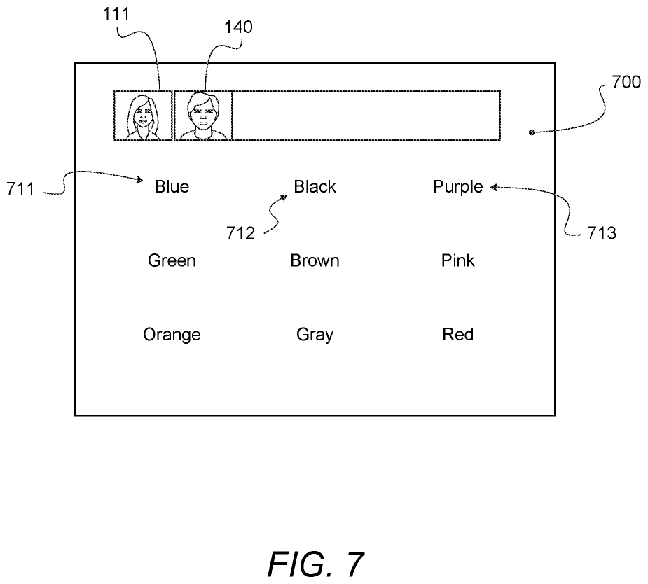

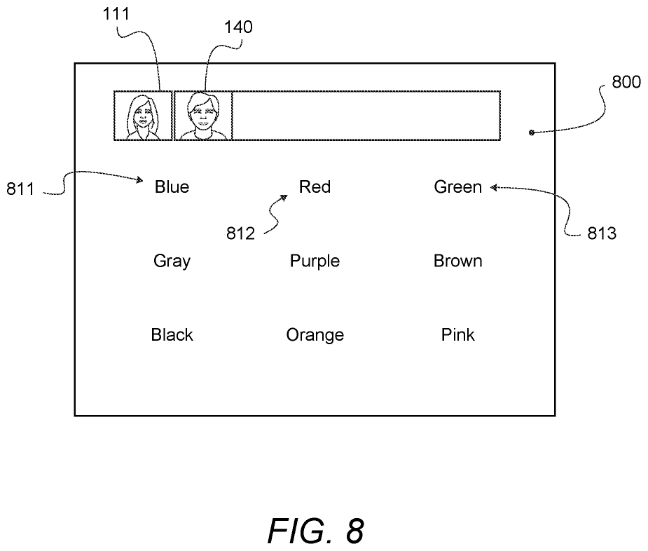

Congruent Stroop task: The congruent Stroop task comprises the user 140 reading a list of colors presented via the computing device 110 in which the text and the colors match. FIG. 7 shows the presentation of the congruent Stroop task 600 on the screen of computing device 110. Though FIG. 6 is in black and white here, in practice each word is colored according to the name of the color. Thus, the word “blue” 611 is colored blue, the word “black” 612 is colored black, the word “purple” 613 is colored purple, and so on. For this task, the camera 112 captures the user's eye movements as they fixate on a word to read and then transition from one word to the next. Audio data corresponding to the user 140 reading a word is also captured via a microphone coupled with computing device 110. The audio data captured is the voice of the user as they read the text. For this task, the server 120 can derive metrics based on a lag between the predicted eye gaze and the speech utterance (e.g., the time at which the user 140 fixates on a certain word and then the time when they actually start speaking out the word) based on a time stamp, a reference frame, or other indicator of time of the predicted eye gaze in the video image data and the speech utterance for the word read from the screen. The greater the lag, the greater the chance of the user 140 suffering from some type of cognitive impairment. Other metrics include the speed of saccade of the user 140 as they change their gaze from one word to the next, the accuracy of the word spoken (whereby the server 120 applies known speech recognition techniques to determine whether the word is the correct one), etc.

Incongruent Stroop task: The incongruent Stroop tasks comprises a presentation of a list of colors via the computing device 110, where the text and the colors do not match. FIG. 8 shows the presentation of the incongruent Stroop task 700 on the screen of computing device 110. Unlike the congruent Stroop task of FIG. 6, here, the colors do not match the text. Thus, the word “blue” 711 is actually colored green, the word “red” 712 is actually colored gray, the word “green” 713 is actually colored black, and so on. The metrics derived by the server 120 for the incongruent Stroop task can the same types of metrics as those derived for the congruent Stroop task discussed above.

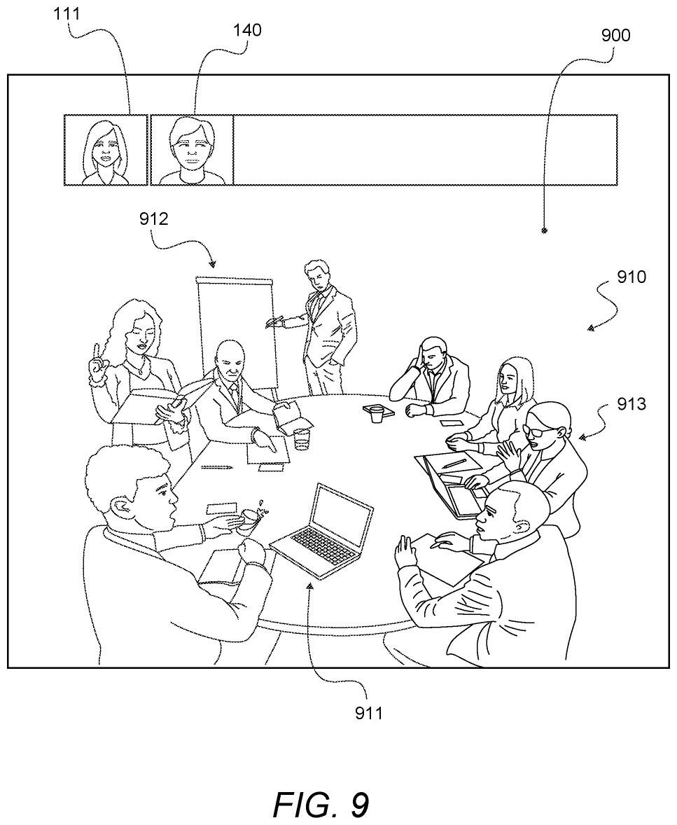

Directed image exploration task: The directed image exploration comprises a presentation of an image, whereby the user 140 is prompted to find and hold their gaze on one or more objects within the image. FIG. 9 shows an illustrative example of the presentation of the directed image exploration task 800 depicting an image 810. For this task the virtual agent 111 could instruct the user 140 to find the laptop computer 811 and hold their gaze on the laptop computer 811 until the virtual agent 111 instructs them to stop or to look at something else in the image 810. For example, after instructing the user 140 to look at the laptop computer 811, the virtual agent 111 can instruct the user to find and fix their gaze on the board 812, and after that the woman wearing glasses 813.

Free image exploration task: for this task, the virtual agent 111 instructs the user 140 to freely look at an image. This can be the same image or a different image used for the direct image exploration task.

As discussed herein, for each task the virtual agent 111 prompts the user 140 with instructions on how to perform the task. The instructions can include audible instructions and/or text instructions. In embodiments, the instructions can include a video demonstration of how the task is to be performed. The virtual agent 111 can indicate the start of the test. The camera 112 captures image data in the form of video data. The task continues until the task has been completed or an allotted amount of time has been spent on the task. The end-point of the task can depend on the task itself. For example, the free image exploration may only have an allotted amount of time limitation without a specific action or objective to be completed.

In embodiments of the inventive subject matter, the tasks 122 can be presented in a set order. For example, calibration, extreme saccade, smooth pursuit line, saccade, congruent Stroop, incongruent Stroop and the image exploration tasks (the directed and free image exploration tasks, discussed above and in further detail below). As the tasks 122 are modular and self-contained within the database 121, the order of the tasks 122 can be modified. However, as noted above, in most embodiments the calibration task will be performed first.

The order of the tasks 122 can be, in embodiments, changed in real-time by the server 120. In another example, the server 120 can change the order of the tasks to account for system limitations at any particular point in time. The server 120 can monitor network performance and other aspects of the network 130 and the user computing device 110 and change the order of the tasks 122 based on a technical issue or problem. To do so, the server 120 is programmed to monitor network conditions and performance and, to the extent possible, keep track of the performance of the user computer device 110 via information gathered by the browser portal and/or the virtual agent 111.

For example, if the server 120 detects a network slowdown, the server 120 can select tasks 122 that are less data-intensive or that require less bandwidth in terms of data obtained (e.g., tasks with video-only requirements as opposed to those with video and audio). In another example, if the browser is out of date or the camera resolution is low, the server 120 can select tasks 122 where the necessary video resolution is lower for the footage to be usable (e.g., extreme saccade where the eye movements are large and the contrast between the pupils and whites of the eyes can serve to track the eye gaze, versus a task where the eye movements are more subtle). In another example, the server 120 can determine that the audio quality is below a usable level, reflecting that a patient's microphone isn't working properly. The server 120 then would select tasks 122 that do not require capture of audio data.

The server 120 can also modify the order of the tasks 122 based on conditions related to the user 140. If certain metrics are more desired for a particular patient, the server 120 can prioritize the tasks 122 that gather those particular metrics first. Other patient-related reasons could include accounting for a patient's fatigue (e.g., if a user's performance in tasks 122 falls off above a certain threshold of degradation). In these cases, the server 120 can select tasks 122 that are less intense or require less effort for the user 140 to perform.

In embodiments, the free image exploration and directed image exploration tasks can be administered as a part of a collective image exploration task in multiple ways.

In one instance, the collective image exploration task can comprise the virtual agent 111 first administering the free image exploration task for a pre-determined amount of time and then administer the directed image exploration task to find a certain number of objects (for example, three specific objects) in the image.

In another instance, the collective image exploration task can comprise the virtual agent 111 first administering the directed image exploration task to find a certain number of objects (for example, three specific objects) in the image and then administering the free image exploration task for a pre-determined amount of time.

At step 240, the camera 112 captures the user's performance of the tasks. As the camera 112 captures the image data, the user computing device 110 segments the image data. The size of the segments can be dependent on the task 122 being performed. For example, for tasks with discrete actions or cycles (such as the smooth pursuit tasks), a segment can be a set amount of cycles (e.g., the line subtask can have a segment that is one cycle (left to right to left along the line 411) long, whereas the circular path subtask can have a segment for each revolution around the circular path 412; the saccade tasks can have segments corresponding to each new appearing object; the directed image exploration task can have a segment corresponding to each new object mentioned that the user must find within the image). For other tasks, such as the Stroop tasks and the free image exploration task, the segment division can be a length of time during the recorded performance of the task. For example, for the Stroop tasks the segment can be a 9-second long segment, whereas for the free image exploration task the segment can be a two-second long segment.

At step 250, the segments of video data stream captured by camera 112 are split into raw video capture data and simultaneous live video processing data across a multitude of operating systems, browser types, screen sizes and resolutions. The raw video capture data segments and live video processing data segments are augmented with facial landmarks using known facial recognition software processes, such as MediaPipe.

The augmented segments of raw video capture data are sent directly to the server 120.

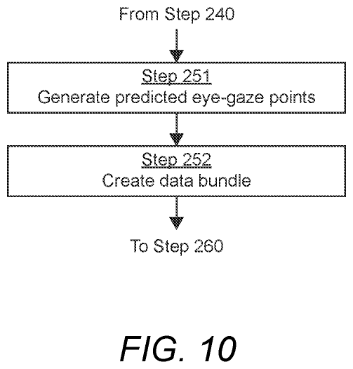

The augmented segments of simultaneous live processing data are processed by the user's computing device 110. The processing of the live processing data is illustrated in the flowchart of FIG. 10.

At step 251, the computing device 110 generates predicted eye-gaze points using known software such as WebGazer. Predicted eye-gaze points are eye-gaze points within one or more frames of the video that are predicted based on prior video data. The computing device 110 also obtains information such as browser information (if the tasks are being executed on a browser) or application information (if the tasks are administered and executed via an installed application), and task information relevant to the task such as a task identifier.

At step 252, the user computing device 110 bundles the live video segment with the predicted eye gaze point data derived for the segment, the browser information for the segment, and the task information for the segment, and sends it to the server 120.

The division of the captured video stream enables the system 100 to account for interruptions of or degradations in network services, whereby the server 120 can receive segments that are ready for additional processing. If a network interruption were to occur, the server 120 can work on the already-received segments for a task while waiting for service to be restored.

For tasks that use a microphone to capture audio data, the audio data is provided directly to the server 120 by the user's computing device 110.

At step 260, the server 120 checks that the data collected for a particular task was properly synchronized.

The synchronization step is critical for the administration and evaluation of eye-gaze-based tasks over a network environment because it addresses potential limitations or challenges in a telemedicine environment that simply would not exist in an in-person setting. In the clinical in-person setting, the communication is direct and thus what the clinician observes visually is directly correlated with what they hear.

In contrast, because the data associated with the performance of the test is captured digitally, transmitted, and then analyzed in order to ascertain the possibility that the user displays symptoms of MND or MCI, there is a danger that unsynchronized data could prevent an accurate diagnosis or return an inaccurate one. In particular, because many of the tasks deal with a user training and moving their eye gaze, a desynchronization in the data could result in a misdiagnosis because a visual lag in the video data could be misinterpreted as a possible sign of MND or MCI.

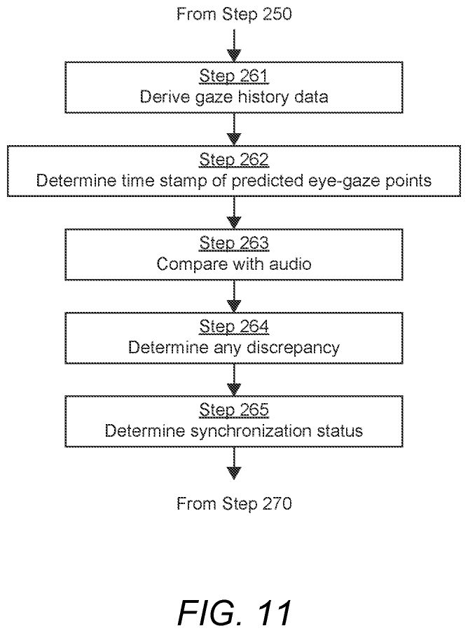

FIG. 11 is a flowchart illustrating the synchronization process of step 260, according to embodiments of the inventive subject matter.

At step 261, the server 120 derives gaze history data from the video data of the segment. The gaze history data is a collection of points across multiple frames of the segment that show where the user's gaze was detected.

At step 262, the server 120 determines the timestamps or other time indicator of the predicted gaze eye-gaze points derived at step 251.

At step 263, the server 120 further compares these with the audio corresponding to the frames used at step 262.

At step 264, the server 120 determines whether there is any discrepancy among the compared reference sets (the predicted eye-gaze points in the history data and the audio data).

The server 120 then determines whether any discrepancy is a difference above a predetermined threshold between the audio data and the predicted eye-gaze points in the history data. If so, the server 120 determines that the data is not synchronized at step 265.

Upon determining that the data is not synchronized for a segment, the server 120 can proceed to check subsequent segments. If a predetermined number of consecutive segments are deemed to not be synchronized (e.g., 3 segments), the server 120 can return a message indicating that the task needs to be repeated at step 265.

In embodiments of the inventive subject matter, the server 120 performs synchronization checks after the completion of the administration and performance of a task 122.

In embodiments of the inventive subject matter, actual gaze data can be obtained with known specialized hardware and used in combination with the predicted eye gaze data and the audio data.

At step 270, the server 120 derives measures for the segments, based on the task 122 being performed. The measures can be considered attributes associated with a particular task. Examples of attributes includes:

General attributes (across all tasks 122): gaze history, fps, canvas width, canvas height, calibration type, history type.

Stimulus Based (Smooth Pursuit, Saccade, Directed Image): stimulus ID, stimulus history.

Stimulus Saccade Based (Saccade, Directed Image): saccade duration.

Stroop (Congruent Stroop, Incongruent Stroop): start utterance frame, end utterance frame (per color).

Image Based (Congruent Stroop, Incongruent Stroop, Directed Image, Free Image): image ID, image origin X, image origin Y, image width, image height

At step 280, the server 120 generates metrics based on the video data (both the raw video data and the live processing data), audio data (if available) and the measures of step 270. When appropriate, metrics are measured along the x-axis, the y-axis and absolute (Euclidean) distance between points. Means, medians, maximums and standard deviations of each variation are then calculated as additional metrics. Examples of metrics includes (in addition to those already mentioned above with regard to some of the individual tasks):

General metrics: Displacement from origin, Velocity (speed of eye gaze movement), Acceleration (acceleration of the eyes as they move from one point to another; deceleration of the eyes as they come to rest on a particular object of focus).

Stroop (Congruent Stroop, Incongruent Stroop): Utterance to saccade delay (as discussed above with regard to the Stroop tasks).

Stimulus Based (Smooth Pursuit, Saccade, Directed Image): Distance from stimulus, Mean Squared Error of distances from stimulus, time to target, time on target, number of times on target.

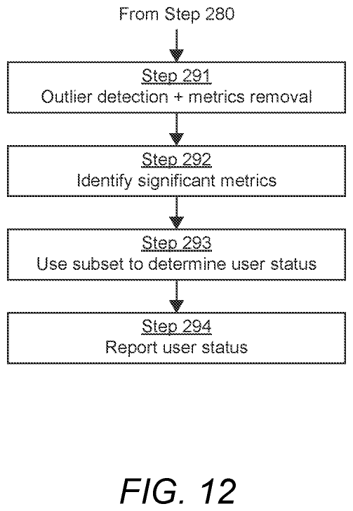

At step 290, the server processes the metrics as shown in the flowchart of FIG. 12.

At this step, demographics data such as age, country of birth, employment status, ethnicity, first language, gender, relationship/marital status, sex and student status can be merged with the quantitative metrics. The use of demographics data can be optional, in embodiments of the inventive subject matter.

At step 291, the server 120 performs outlier detection and removal of objective metrics according to the following process:

First, all objective metrics beyond five standard deviations of their distributions are removed. Then, for the remaining values, the mean and standard deviation of the distribution are recalculated and all values beyond three standard deviations are removed.

At step 292, the server 120 applies statistical tests (e.g., Pearson correlation or other feature selection methods) to identify metrics that are significant in distinguishing differences between people that, in a priori data gathering, Mild Neurocognitive Disorder (MND)/Mild Cognitive Impairment (MCI) compared to those who have not. The metrics are scored and ranked accordingly. The ranking can be performed by the server 120 according to a number of ranking criteria, including but not limited to: (i) capturing differences between cohorts like MCI vs results from control groups; (ii) low correlation between features groups selected (so that they are each capturing different pieces of information); (iii) ease and fidelity of extraction across languages and technologies, etc.

A Pearson correlation returns a number score for metrics for based on impactful they are, for distinguishing differences with other metrics. In this case, the system can see how each measure and metric impact the existence of MND in a user. Thus, a higher score for a metric means higher correlation to the problem at hand.

At step 293, a subset of the metrics which are above a threshold score (determined by performance of the metrics against a number of frequently-used, high-performing classifiers) are used to determine the user status-whether a user is likely or not to have MND or MCI, or another cognitive impairment. In an example, the server 120 can run different subsets of the metrics using various thresholds using known machine learning methods made for classification. The server 120 then analyzes the general performance of each threshold and selects the best-performing metrics (e.g., a pre-defined amount of best performing metrics).

To determine whether a user 140 is exhibiting symptoms or signs of MND or MCI, the server 120 can, in embodiments of the inventive subject matter, apply the collected metrics and their values against metric profiles for MND or MCI, or other cognitive impairment. A metric profile can be considered to be a collection of metrics and threshold values for those metrics that, when exceeded, indicate a likely presence of a condition (MND, MCI, or other impairment represented by the metric profile). The degree of the condition can be determined by the server 120 based the extent to which the measured metrics exceed the metrics in the metric profiles. It is contemplated that the metrics within a particular metric profile can be weighted based on the particular impairment represented by the metric profile. Thus, to determine which (if any) conditions a user 140 may be suffering from, the server 120 applies the derived metrics from the administration of the tasks 122 to the metric profiles of some or all of the available conditions.

At step 294, the determined user status/condition can be reported. The server 120 can report the condition back to the user 140 via the user computing device 110 by way of the virtual agent 111 or other presentation. The determined condition can also be reported to healthcare providers, insurance providers, or other interested parties for the care and continued planning for the user 140.

In embodiments of the inventive subject matter the server 120 can execute a new set of one or more of the tasks 122 for the user 140 based on the results of step 293. For example, if the results mark a significant difference from past performances of the tasks, the server 120 can inform the user 140 via the virtual agent 111 that additional tasks need to be performed. The server 120 can determine this by comparing the results of the newest performance against historical performance of the tasks. If the variance is above a threshold amount, the server 120 flags it as a potential anomaly and re-executes the tasks accordingly.

The server 120 can, in embodiments of the inventive subject matter, re-run tasks 122 based on results affected by a detected desynchronization as discussed above. A desynchronized task 122 can cause a “hole” in data gathered during a task such that one or more of the desired metrics cannot be derived. In this situation, the server 120 can re-run the tasks 122 affected by the desynchronization to gather usable image and audio data from the user 140.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method for remote patient assessment via eye tracking, comprising:
presenting, by a virtual agent and via a screen, a visual task for a user to perform;
capturing, by at least one processor and via a camera, image data of the user performing the visual task, wherein the image data depicts eye activity corresponding to the user's eyes while performing the visual task;
predicting, by the at least one processor, at least one predicted eye gaze position for at least one time during the performance of the visual task based on the captured image data;
determining, by the at least one processor, a synchronization status of either synchronized or unsynchronized for the visual task based on the at least one predicted eye gaze position for the at least one time and a timestamp of the captured image data;
in response to determining that the synchronization status is synchronized, performing, by the at least one processor, visual analysis of the eye activity depicted in the image data according to at least one measure;
deriving, by the at least one processor, at least one metric for the eye activity based on the at least one measure;
correlating, by the at least one processor, the at least one metric to at least one patient condition; and
returning, by the at least one processor, a determination of a condition among the at least one patient condition based on the correlation.

2. The method of claim 1, further comprising prior to the presenting step, executing, by the at least one processor, a calibration task.

3. The method of claim 2, wherein the calibration task comprises:
presenting, by the at least one processor, a display with a plurality of points;
prompting, by the at least one processor, the user to navigate a mouse cursor to each of the plurality of points in a predetermined sequence;
detecting, by the at least one processor, a movement of a mouse cursor toward each of the plurality of points;
receiving, by the at least one processor, calibration image data, wherein the calibration image data comprises a depiction of eye movements of the user as the user move the mouse cursor during the calibration task;
correlating, by the at least one processor, the detected movement of the mouse cursor with the depiction of the eye movements; and
establish, by the at least one processor, task boundaries based on the correlation, wherein the task boundaries define a usable area within which the visual task is to be presented.

4. The method of claim 1, further comprising, prior to the presenting step, a normalization task, wherein the normalization task comprises:
receiving, by the at least one processor, the image data, wherein the image data depicts the face of the user;
overlaying, by the at least one processor, a box on the image data, such that the face is centered within the box and such that the edges of the face at least touch edges of the box; and
displaying, by the at least one processor, the box overlay during the performance of the visual task.

5. The method of claim 4, wherein the normalization task further comprises:
obtaining, by the at least one processor, display information about a display on which the visual task is to be presented;
determining, a resolution of a usable area based on the display information; and
obtaining, by the at least one processor, task information associated with the visual task, wherein the task information includes at least one of a spacing ratio, an object size information, an object location information, and a movement information;
wherein the presenting of the visual task is based on the task information and the resolution of the usable area and wherein the at least one metric is derived based on the task information and the display information.

6. The method of claim 1, wherein visual task comprises at least one of an extreme saccade task, a saccade task, a smooth pursuit task, a congruent Stroop task, an incongruent Stroop task, a directed image exploration task, and a free image exploration task.

7. The method of claim 1, where the at least one metric comprises an amount of eye movement, a smoothness of eye movement, a gaze sequence, a saccade, and a fixation.

8. The method of claim 1, wherein the determination of a condition comprises:
comparing, by the at least one processor, the at least one metric to a corresponding at least one patient condition metric, the patient condition metric having a threshold value;
determining, by the at least one processor, that a value of the at least one metric exceeds the threshold value; and
returning, by the at least one processor, the determination of the condition based on the determination that the value of the at least one metric exceeds the threshold value.

9. A system for remote patient assessment via eye tracking, comprising:
a server comprising a processor and a database, the database storing at least one visual task for a user to perform;
a user computing device communicatively coupled with the server, the user computing device programmed to:
present, by a visual agent via a display screen, the at least one visual task;
capture, via a camera coupled to the user computer device, image data of the user performing the visual task, wherein the image data depicts eye activity corresponding to the user's eyes while performing the visual task;
transmit the captured image data to the server; and
present the user status via the display screen; and
wherein the server is programmed to:
receive the image data;
predict at least one predicted eye gaze position for at least one time during the performance of the visual task based on the captured image data;
determine a synchronization status of either synchronized or unsynchronized for the visual task based on the at least one predicted eye gaze position for the at least one time and a timestamp of the captured image data;
in response to determining that the synchronization status is synchronized, perform a visual analysis of the eye activity depicted in the image data according to at least one measure;
derive at least one metric for the eye activity based on the at least one measure;
correlate the at least one metric to at least one patient condition; and
return a determination of a condition among the at least one patient condition based on the correlation.

10. The system of claim 9, wherein the user computing device is programmed to, prior to presenting the at least one visual task, present a calibration task via the display screen.

11. The system of claim 10, wherein the calibration task comprises at least one of the user computing device and the server programmed to:
present a display with a plurality of points;
prompt the user to navigate a mouse cursor to each of the plurality of points in a predetermined sequence;
detect a movement of a mouse cursor toward each of the plurality of points;
receive, calibration image data, wherein the calibration image data comprises a depiction of eye movements of the user as the user move the mouse cursor during the calibration task;
correlate the detected movement of the mouse cursor with the depiction of the eye movements; and
establish task boundaries based on the correlation, wherein the task boundaries define a usable area within which the visual task is to be presented.

12. The system of claim 9, further comprising the user computing device programmed to, prior to the presenting step, present a normalization task, wherein the normalization task comprises at least one of the user computing device and the server programmed to:
receive the image data, wherein the image data depicts the face of the user;
overlay a box on the image data, such that the face is centered within the box and such that the edges of the face at least touch edges of the box; and
display the box overlay during the performance of the visual task.

13. The system of claim 12, wherein the normalization task further comprises at least one of the user computing device and the server further programmed to:
obtain display information about a display on which the visual task is to be presented;
determine a resolution of a usable area based on the display information; and
obtain task information associated with the visual task, wherein the task information includes at least one of a spacing ratio, an object size information, an object location information, and a movement information;
wherein the presenting of the visual task is based on the task information and the resolution of the usable area and wherein the at least one metric is derived based on the task information and the display information.

14. The system of claim 9, wherein visual task comprises at least one of an extreme saccade task, a saccade task, a smooth pursuit task, a congruent Stroop task, an incongruent Stroop task, a directed image exploration task, and a free image exploration task.

15. The system of claim 9, wherein the at least one metric comprises an amount of eye movement, a smoothness of eye movement, a gaze sequence, a saccade, and a fixation.

16. The system of claim 9, wherein the determination of a condition comprises the server programmed to:
compare the at least one metric to a corresponding at least one patient condition metric, the patient condition metric having a threshold value; and
determine that a value of the at least one metric exceeds the threshold value; and
return the determination of the condition based on the determination that the value of the at least one metric exceeds the threshold value.
